(19) United States
(12) Patent Application Publication
(10) Pub. No.: US 2022/0277806 A1
(43) Pub. Date: Sep. 1, 2022

(54) SYSTEM AND METHOD FOR PROVIDING GENETIC SURNAME INFORMATION BY USING GENOMIC INFORMATION

(71) Applicant: CLINOMICS INC., Ulsan (KR)

(72) Inventors: Jong Hwa BHAK, Ulsan (KR); Yun Sung CHO, Yongin-si (KR); Byung Chul KIM, Yongin-si (KR)

(21) Appl. No.: 17/746,753

(22) Filed: May 17, 2022

Related U.S. Application Data
(63) Continuation-in-part of application No. PCT/KR2019/017532, filed on Dec. 12, 2019.

(30) Foreign Application Priority Data
Dec. 6, 2019 (KR) 10-2019-0161750

(51) Int. Cl. G16B 20/40; G16B 30/10; C12Q 1/6869; C12Q 1/6876

(52) U.S. Cl. CPC G16B 20/40; G16B 30/10; C12Q 1/6869; C12Q 1/6876

(57) ABSTRACT

According to an embodiment, a system for providing genetic surname information using genomic information may include a Y-chromosome grouping unit, a male genetic surname determination unit, and a male genetic surname information providing unit and may further include an X-chromosome grouping unit and a female genetic surname determination unit.

### CROSS-REFERENCE TO RELATED APPLICATION(S)

This U.S. non-provisional application is a continuation-in-part of International Patent Application No. PCT/KR2019/017532, filed on Dec. 12, 2019, which claims priority to Korean Patent Application No. 10-2019-0161750 filed in the Korean Intellectual Property Office on Dec. 6, 2019, the disclosures of which are incorporated by reference herein in their entireties.

### TECHNICAL FIELD

Embodiments of the present invention relate to a system and method for providing genetic surname information using genomic information.

### DISCUSSION OF RELATED ART

The surname used these days is presumed to have been used since the Silla Dynasty. The legends for the three surnames Park, Seok, and Kim have been passed down, and it has been known that in the 9th year of King Yuri, the surnames Lee, Choi, Son, Jeong, Bae, and Seol were given to the village chiefs of the six clans.

In the 9th year of King Munjong (AD 1055) in the middle of the Goryeo Dynasty, the use of surnames became common with the promulgation of a decree stating that those without a surname were not entitled to take the imperial exam.

In the early days of the Joseon Dynasty, the use of surnames was common to the commoners but was not allowed for the low-class people, such as slaves.

In the modern era, with the enforcement of the Family Register Act in 1909, the law was enacted so that anyone could have a surname, so that all Koreans could use their surname.

At that time, surnames were created for people who did not have one, and people who were in charge of the family register made anyone who desires a surname.

It may be shown that the surname was given arbitrarily without any relation by blood.

As a result, the number of surnames has increased a lot. Under Japanese colonial rule, there was once again great confusion due to Sōshi-kaimei as a part of the japanization policy and the Restoration of the Korean Names promulgated by the U.S. Military Government in 1946.

In most countries including Korea, the family name (or ‘surname’) is inherited patrilineally from one's father, but such an issue arises where, from generation to generation, surnames do not perfectly match genes.

In particular, it is very unclear whether one's surname belongs to a surname group of a specific gene for various reasons, such as selling and buying the genealogy book, adoptions, adultery, etc.

### SUMMARY

Embodiments of the present invention provide a system and method for providing genetic surname information using genomic information, which define the Y-chromosome group where a male himself belongs and sub groups thereof, precisely calculate the degree of genetic variation to the Y-chromosome to thereby infer the genetic surname closest to the male, such as siblings, ancestors, cousins, first cousins once removed, or first cousins four times removed, and provide a female's surname by comparing the autosomal, X-chromosomal, and mitochondrial DNA information possessed by the Y-chromosome-based genetic surname group and the female's autosomal, X-chromosomal, and mitochondrial DNA information, using variation information about the autosomal, X-chromosomal, and mitochondrial DANs present in both the male and female.

According to an embodiment of the present invention, a system for providing genetic surname information using genomic information comprises a Y-chromosome grouping unit identifying genetic variations of Y-chromosomes for ancient and modern males in each area or country, grouping the Y-chromosomes for the ancient and modern males based on the identified genetic variations, and if a specific surname for the males grouped according to the genetic variations is shown in a predetermined proportion or more, specifying a corresponding group as a Y-chromosome group and storing the Y-chromosome group in a Y-chromosome group database, a male genetic surname determination unit mapping a genetic marker for determining a male client's Y-chromosome to a genetic marker in a Y-chromosome in each Y-chromosome group stored in the Y-chromosome group database to thereby calculate a genetic distance and determining that a surname of a Y-chromosome group having the genetic distance closest to the male client's Y-chromosome is the male client's genetic surname, and a male genetic surname information providing unit providing the male client with genetic surname information including at least one of social information, historical information, emotional information, and physical information about the modern or ancient males having the genetic surname determined through the male genetic surname determination unit.

Further, the Y-chromosome grouping unit may include a Y-chromosome genetic variation detection unit analyzing the genetic information about the Y-chromosomes for the ancient and modern males in each area or country, comparing sequences of the Y-chromosomes for which the genetic information has been analyzed, and detecting genetic variations of the Y-chromosomes from a result of the comparison, a Y-chromosome marker designation unit mutually comparing the genetic variations detected through the Y-chromosome genetic variation detection unit and designating a marker for determining a Y-chromosome according to a distance of the Y-chromosome on a map, and a Y-chromosome clustering unit grouping the Y-chromosomes for the ancient and modern males and, if the specific surname for the males grouped according to the genetic variations of the Y-chromosomes occupies ⅓ or more of all surnames, specifying the corresponding group as the Y-chromosome group, and storing the Y-chromosome group in the Y-chromosome group database.

Further, the male genetic surname determination unit may include a genetic surname similarity calculation unit mapping the genetic marker for determining the male client's Y-chromosome to the genetic marker of the Y-chromosome in each Y-chromosome group stored in the Y-chromosome group database to thereby calculate the genetic distance, and scoring a similarity between the male client and the surnames belonging to each Y-chromosome group and a genetic surname prediction unit predicting a surname of a Y-chromosome group having a highest similarity score with the male client's Y-chromosome through the genetic surname similarity calculation unit, as the male client's genetic surname.

Further, the male genetic surname information providing unit may provide social information about a figure known to the public among the males belonging to the genetic surname determined through the male genetic surname determination unit, historical information including past migration and settlement route information about the males belonging to the surname discovered in the Y-chromosome group of the genetic surname, emotional information including personality of the males belonging to the genetic surname, and physical information including body features, blood types, and diseases of the males belonging to the genetic surname.

Further, the system may further comprise an X-chromosome grouping unit identifying genetic variations of autosomal, X-chromosomal and mitochondrial DNAs of the males belonging to the Y-chromosome group, combining the autosomal, X-chromosomal and mitochondrial DNAs with the same or most similar distribution patterns of the identified genetic variations, specifying the combined autosomal, X-chromosomal and mitochondrial DNAs as an X-chromosome group, and storing the X-chromosome group in an X-chromosome group database and a female genetic surname determination unit mapping a genetic marker for determining a female client's X-chromosome to a genetic marker in an X-chromosome in each X-chromosome group stored in the X-chromosome group database to thereby calculate a genetic distance and determining that a surname of an X-chromosome group having the genetic distance closest to the client's X-chromosome is the client's genetic surname. The male genetic surname information providing unit may provide the client with genetic surname information including at least one of social information, historical information, emotional information, and physical information about the modern or ancient males having the genetic surname determined through the female genetic surname determination unit.

According to another embodiment of the present invention, a method for providing genetic surname information using genomic information comprises a Y-chromosome grouping step identifying genetic variations of Y-chromosomes for ancient and modern males in each area or country, grouping the Y-chromosomes for the ancient and modern males based on the identified genetic variations, and if a specific surname for the males grouped according to the genetic variations is shown in a predetermined proportion or more, specifying a corresponding group as a Y-chromosome group and storing the Y-chromosome group in a Y-chromosome group database, by a Y-chromosome grouping unit, a male genetic surname determination step mapping a genetic marker for determining a male client's Y-chromosome to a genetic marker in a Y-chromosome in each Y-chromosome group stored in the Y-chromosome group database to thereby calculate a genetic distance and determining that a surname of a Y-chromosome group having the genetic distance closest to the male client's Y-chromosome is the male client's genetic surname, by a male genetic surname determination unit, and a male genetic surname information providing step providing the male client with genetic surname information including at least one of social information, historical information, emotional information, and physical information about the modern or ancient males having the genetic surname determined through the male genetic surname determination step, by a male genetic surname information providing unit.

Further, the Y-chromosome grouping unit step may include a Y-chromosome genetic variation detection step analyzing the genetic information about the Y-chromosomes for the ancient and modern males in each area or country, comparing sequences of the Y-chromosomes for which the genetic information has been analyzed, and detecting genetic variations of the Y-chromosomes from a result of the comparison, a Y-chromosome marker designation step mutually comparing the genetic variations detected through the Y-chromosome genetic variation detection step and designating a marker for determining a Y-chromosome according to a distance of the Y-chromosome on a map, and a Y-chromosome clustering step grouping the Y-chromosomes for the ancient and modern males and, if the specific surname for the males grouped according to the genetic variations of the Y-chromosomes occupies ⅓ or more of all surnames, specifying the corresponding group as the Y-chromosome group, and storing the Y-chromosome group in the Y-chromosome group database.

Further, the male genetic surname determination step may include a genetic surname similarity calculation step mapping the genetic marker for determining the male client's Y-chromosome to the genetic marker of the Y-chromosome in each Y-chromosome group stored in the Y-chromosome group database to thereby calculate the genetic distance, and scoring a similarity between the male client and the surnames belonging to each Y-chromosome group and a genetic surname prediction step predicting a surname of a Y-chromosome group having a highest similarity score with the male client's Y-chromosome through the genetic surname similarity calculation step, as the male client's genetic surname.

Further, the male genetic surname information providing step may provide social information about a figure known to the public among the males belonging to the genetic surname determined through the male genetic surname determination step, historical information including past migration and settlement route information about the males belonging to the surname discovered in the Y-chromosome group of the genetic surname, emotional information including personality of the males belonging to the genetic surname, and physical information including body features, blood types, and diseases of the males belonging to the genetic surname.

Further, the method may further comprise an X-chromosome grouping step identifying genetic variations of autosomal, X-chromosomal and mitochondrial DNAs of the males belonging to the Y-chromosome group, combining the autosomal, X-chromosomal and mitochondrial DNAs with the same or most similar distribution patterns of the identified genetic variations, specifying the combined autosomal, X-chromosomal and mitochondrial DNAs as an X-chromosome group, and storing the X-chromosome group in an X-chromosome group database, by an X-chromosome grouping unit and a female genetic surname determination step mapping a genetic marker for determining a female client's X-chromosome to a genetic marker in an X-chromosome in each X-chromosome group stored in the X-chromosome group database to thereby calculate a genetic distance and determining that a surname of an X-chromosome group having the genetic distance closest to the client's X-chromosome is the client's genetic surname, by a female genetic surname determination unit. The male genetic surname information providing step may provide the client with genetic surname information including at least one of social information, historical information, emotional information, and physical information about the modern or ancient males having the genetic surname determined through the female genetic surname determination step.

According to the present invention, there may be provided a system and method for providing genetic surname information using genomic information, which define the Y-chromosome group where a male himself belongs and sub groups thereof, precisely calculate the degree of genetic variation to the Y-chromosome to thereby infer the genetic surname closest to the male, such as siblings, ancestors, cousins, first cousins once removed, or first cousins four times removed, and provide a female's surname by comparing the autosomal, X-chromosomal, and mitochondrial DNA information possessed by the Y-chromosome-based genetic surname group and the female's autosomal, X-chromosomal, and mitochondrial DNA information, using variation information about the autosomal, X-chromosomal, and mitochondrial DANs present in both the male and female.

### BRIEF DESCRIPTION OF THE DRAWINGS

FIG. 1 is a schematic diagram illustrating an overall operational flow of a system for providing genetic surname information using genomic information according to an embodiment of the present invention.

FIG. 2 is a block diagram illustrating an overall configuration of a system for providing genetic surname information using genomic information according to an embodiment of the present invention.

FIG. 3 is a block diagram illustrating a detailed configuration of a Y-chromosome grouping unit according to an embodiment of the present invention.

FIG. 4 is a block diagram illustrating a detailed configuration of a male genetic determination unit according to an embodiment of the present invention.

FIG. 5 is a flowchart illustrating an overall process of a method for providing genetic surname information using genomic information according to an embodiment of the present invention.

FIG. 6 is a flowchart illustrating a detailed process of a Y-chromosome grouping step according to an embodiment of the present invention.

FIG. 7 is a flowchart illustrating a detailed process of a male genetic surname determination step according to an embodiment of the present invention.

### DETAILED DESCRIPTION

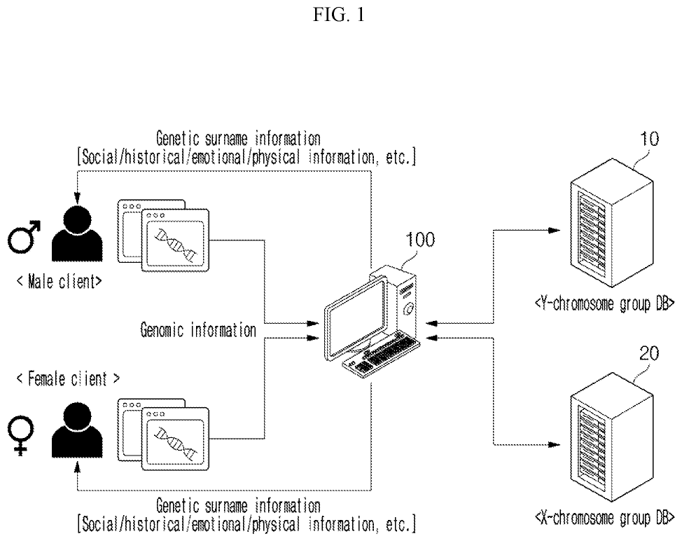

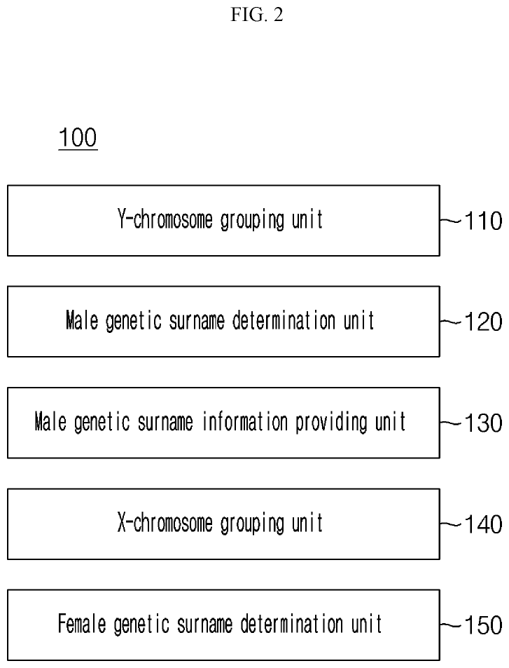

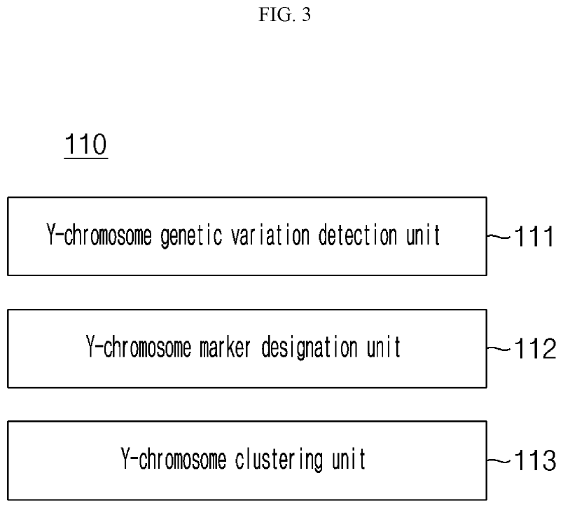

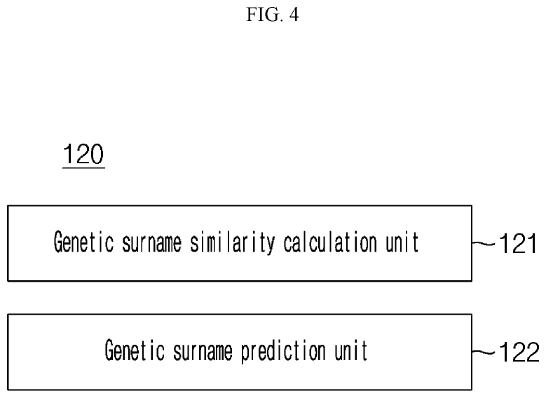

FIG. 1 is a schematic diagram illustrating an overall operational flow of a system for providing genetic surname information using genomic information according to an embodiment of the present invention. FIG. 2 is a block diagram illustrating an overall configuration of a system for providing genetic surname information using genomic information according to an embodiment of the present invention. FIG. 3 is a block diagram illustrating a detailed configuration of a Y-chromosome grouping unit according to an embodiment of the present invention. FIG. 4 is a block diagram illustrating a detailed configuration of a male genetic determination unit according to an embodiment of the present invention.

Referring to FIGS. 1 and 2, a system 100 for providing genetic surname information using genomic information according to an embodiment of the present invention may include a Y-chromosome grouping unit 110, a male genetic surname determination unit 120, and a male genetic surname information providing unit 130 and may further include an X-chromosome grouping unit 140 and a female genetic surname determination unit 150.

Each of the components, e.g., those components denoted as units, may include, or be implemented as, an electrical circuit or circuitry configured to perform its corresponding functions or operations. According to an embodiment, the units may be integrated into, or implemented as, a processor or controller (not shown) configured to perform the respective functions or operations thereof. The functions or operations performed by the units may be programmed into code, instructions, or commands that may be stored in a memory (not shown) and that, when executed by a processor or controller (not shown), enable the processor or controller to perform the functions or operations of the units.

The genetic surname information providing system 100 according to the embodiment may be implemented in a composite form of a gene analyzing device and a program installed on the gene analyzing device and may be connected with a client's communication terminal through a communication network to provide various pieces of information related to genetic surnames to the client's communication terminal.

The Y-chromosome grouping unit 110 may identify genetic variations of the Y-chromosome for ancient and modern males in each area or country, group the Y-chromosomes for the ancient and modern males, in a hierarchical form, based on the identified genetic variations, and if a specific surname for the males grouped according to the genetic variations is shown in a predetermined proportion or more, specify the corresponding group as a Y-chromosome group and store the Y-chromosome group in a Y-chromosome group database 10. To that end, as shown in FIG. 3, the Y-chromosome grouping unit 110 may include a Y-chromosome genetic variation detection unit 111, a Y-chromosome marker designation unit 112, and a Y-chromosome clustering unit 113.

The Y-chromosome genetic variation detection unit 111 may analyze genetic information about the Y-chromosomes for the ancient and modern males in each area or country, compares the sequences of the Y-chromosomes for which genetic information has been analyzed, and detect a genetic variation of the Y-chromosome from the result of the comparison. In order for the Y-chromosome grouping unit 110 to ultimately build the Y-chromosome group database 10, the Y-chromosome genetic variation detection unit 111 performs general processes, such as genome/DNA analysis, mapping to a standard genome map, and variant calling. In this case, the embodiments are not limited for a specific configuration for algorithms and devices for analyzing the genome, chromosome, and DNA or algorithms and devices for extracting genetic variation information later.

The Y-chromosome marker designation unit 112 may mutually compare the genetic variations detected through the Y-chromosome genetic variation detection unit 111 while Y-chromosomes and the surname information for the corresponding man are steadily accumulated in the Y-chromosome group database 10 and may designate a marker for determining the Y-chromosome depending on the distance on the Y-chromosome map.

For example, a database, i.e., the Y-chromosome group database 10, may be built for the Y-chromosomes for 2,000 males having various surnames and their surnames, and a specific marker may be designated for a corresponding chromosome based on the genetic variation information about the Y-chromosome. In this case, the marker has all frequencies between 0.01% and 99% of the occurrence frequency in the population, and the markers for the cases where all people have and it occurs in very few cases, e.g., 0.01%, may be excluded.

The Y-chromosome clustering unit 113 may group the Y-chromosomes for the ancient and modern males and, if a specific surname for the males grouped according to the genetic variations in Y-chromosome occupy ⅓ or more of all the surnames, specify the corresponding group as a Y-chromosome group and store it in the Y-chromosome group database 10.

In the instant embodiment, the Y-chromosome group means a group clustered when a general clustering algorithm and phylogenetic diagram are drawn in the population, and when there is a group in which the surname of the males belonging to the cluster occupies 33% or more among such groups, the group may be specified as the Y-chromosome group. Here, 33% means that a specific surname occupies ⅓ of the population. Besides, depending on each surname, an empirically adjusted number, which is variable, such as 40% or 50%, may be used. As such, without limited to a specific reference value, it is important to be able to cluster into empirically most of Y-chromosome groups.

Meanwhile, if Y-chromosome groups are unable to be clearly distinguished per surname (i.e., when an overlap occurs between the surnames), such a process may be performed as to designate a representative indicator (one or a few) for each surname, positioned in the center or middle of each per-surname distribution to clearly process the position of each surname on the cluster.

The male genetic surname determination unit 120 may map the genetic marker for determining the male client's Y-chromosome to the genetic marker of the Y-chromosome in each Y-chromosome group stored in the Y-chromosome group database 10 to thereby calculate the genetic distance and determine (actually predict or estimate) that the surname of the Y-chromosome group having the genetic distance closest to the male client's Y-chromosome is the male client's genetic surname. To that end, the male genetic surname determination unit 120 may include a genetic surname similarity calculation unit 121 and a genetic surname prediction unit 122 as shown in FIG. 4.

The genetic surname similarity calculation unit 121 may map the genetic marker for determining the male client's Y-chromosome to the genetic marker of the Y-chromosome in each Y-chromosome group stored in the Y-chromosome group database 10 to thereby calculate each genetic distance, score the similarity between the male client's and the surnames belonging to each Y-chromosome group, and provide a numerical result. Here, the genetic surname similarity calculation unit 121 may use an algorithm (including all common algorithms for calculating genetic distance) that maps the marker for determining the male client's Y-chromosome based on the Y-chromosome group database 10 and calculates the distance closest to the Y-chromosome group in the population (e.g., 2,000 people). According to the general concept of the algorithm for calculating genetic distance, each male client has a genetic distance of Y-chromosome for each surname. The genetic distance of Y-chromosome may thus be represented as a score indicating how close to the Y-chromosome group each individual's surname is.

In this case, according to the general concept of calculating the genetic distance of Y-chromosome group, each Y-chromosome group has a database for migration of Y-chromosome group through comparison between a Y-chromosome group database for ancient males and a Y-chromosome group database for international males. Such migration database may become a distinguishing marker indicating how males in the Y-chromosome group have migrated from Africa.

The genetic surname prediction unit 122 may predict the surname of the Y-chromosome group having the highest similarity score to the Y-chromosome of the male client through the genetic surname similarity calculation unit 121, as the male client's genetic surname.

The male genetic surname information providing unit 130 may provide the male client with genetic surname information including at least one of social information, historical information, emotional information, and physical information about the modern or past males having the genetic surname determined through the male genetic surname determination unit 120.

More specifically, the male genetic surname information providing unit 130 may provide each of social information about figures known to the public among the males belonging to the genetic surname determined through the male genetic surname determination unit 120, historical information including past migration and settlement route information about the males belonging to the surname discovered in the Y-chromosome group of the corresponding genetic surname, emotional information including the personality of the males belonging to the genetic surname, and physical information including body characteristics, blood type and disease about the males belonging to the genetic surname.

The male genetic surname information providing unit 130 may provide various pieces of statistical information, such as famous figures (historically) in the present or past among the males belonging to the genetic surname determined through the male genetic surname determination unit 120, historical and social information about the figures, and distribution of surnames discovered in the Y-chromosome group. Further, the male genetic surname information providing unit 130 may provide entertainment information related to anecdotes or characteristics for the surnames of the Y-chromosome group based on the Y-chromosome group. Further, the male genetic surname information providing unit 130 may provide various pieces of information, such as personality, body feature, or blood type of individuals based on the Y-chromosome group. Further, the male genetic surname information providing unit 130 may provide information about the migration or settlement route of ancestors of the Y-chromosome group based on the Y-chromosome group. Such information may be manufactured and provided as image information by way of media through an application and web.

The X-chromosome grouping unit 140 may identify the genetic variation of the autosomal, X-chromosomal and mitochondrial DNAs of the males belonging to the Y-chromosome group, combine the autosomal, X-chromosomal and mitochondrial DNAs with the same or most similar distribution patterns of identified genetic variations, specify the combined autosomal, X-chromosomal and mitochondrial DNAs as an X-chromosome group and store it in the X-chromosome group database 20. The X-chromosome grouping unit 140 is configured to find females' surnames based on the genetic information about the autosomal, X-chromosomal and mitochondrial DNAs of people belonging to the Y-chromosome group in a state in which the Y-chromosome group database 10 is built. The X-chromosome grouping unit 140 has substantially the same configuration as that of the Y-chromosome grouping unit 110, and a detailed description thereof is thus omitted.

The female genetic surname determination unit 150 may map the genetic marker for determining the female client's X-chromosome to the genetic marker of the X-chromosome in each X-chromosome group stored in the X-chromosome group database 20 to thereby calculate the genetic distance and determine that the surname of the X-chromosome group having the genetic distance closest to the client's X-chromosome is the client's genetic surname. The female genetic surname determination unit 150 has substantially the same configuration as that of the male genetic surname determination unit 120, and a detailed description thereof is thus omitted.

Accordingly, the male genetic surname information providing unit 130 may provide the client with genetic surname information including at least one of social information, historical information, emotional information and physical information about the modern or ancient males determined through the female genetic surname determination unit 150 and the configuration therefor is performed in the same manner as the operation of the male genetic surname information providing unit 130 described above.

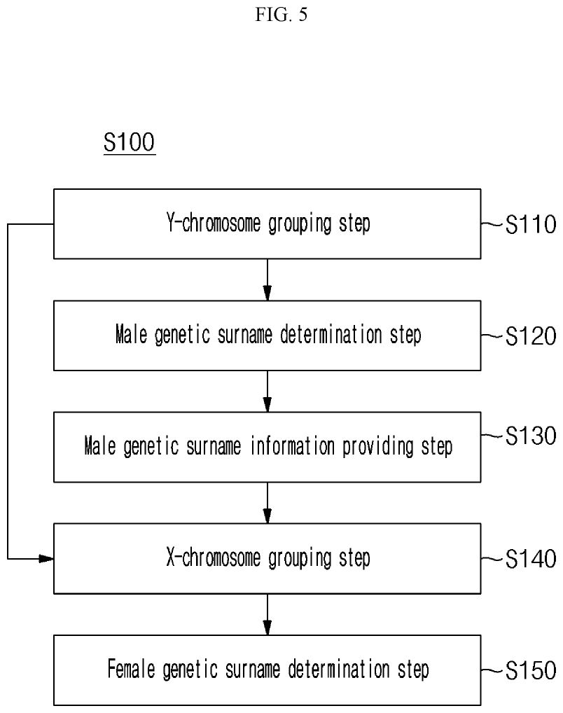

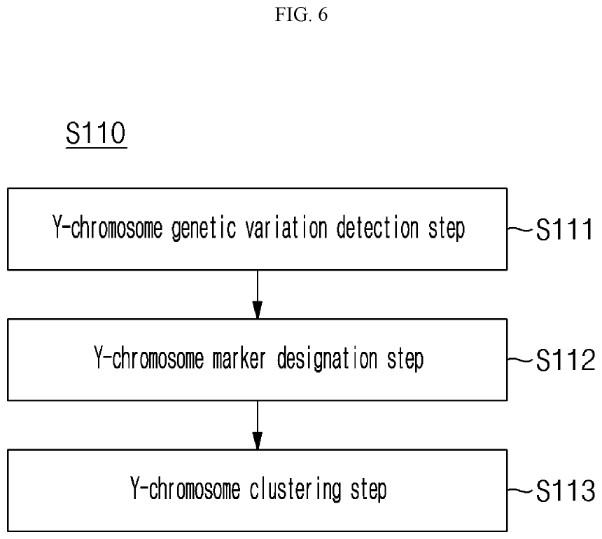

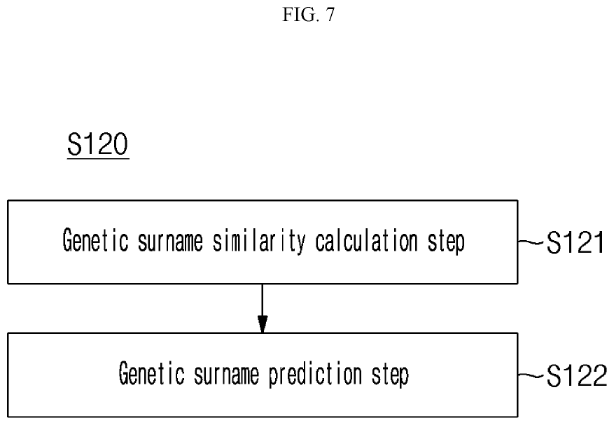

FIG. 5 is a flowchart illustrating an overall process of a method for providing genetic surname information using genomic information according to an embodiment of the present invention. FIG. 6 is a flowchart illustrating a detailed process of a Y-chromosome grouping step according to an embodiment of the present invention. FIG. 7 is a flowchart illustrating a detailed process of a male genetic surname determination step according to an embodiment of the present invention.

Referring to FIG. 5, a method S100 for providing genetic surname information using genomic information according to an embodiment of the present invention may include a Y-chromosome grouping step S110, a male genetic surname determination step S120, and a male genetic surname information providing step S130 and may further include an X-chromosome grouping step S140 and a female genetic surname determination step S150.

The Y-chromosome grouping step S110 may identify genetic variations of the Y-chromosome for ancient and modern males in each area or country, group the Y-chromosomes for the ancient and modern males, in a hierarchical form, based on the identified genetic variations, and if a specific surname for the males grouped according to the genetic variations is shown in a predetermined proportion or more, specify the corresponding group as a Y-chromosome group and store the Y-chromosome group in a Y-chromosome group database 10. To that end, as shown in FIG. 6, the Y-chromosome grouping step S110 may include a Y-chromosome genetic variation detection step S111, a Y-chromosome marker designation step S112, and a Y-chromosome clustering step S113.

The Y-chromosome genetic variation detection step S111 may analyze genetic information about the Y-chromosomes for the ancient and modern males in each area or country, compares the sequences of the Y-chromosomes for which genetic information has been analyzed, and detect a genetic variation of the Y-chromosome from the result of the comparison. In order for the Y-chromosome grouping step S110 to ultimately build the Y-chromosome group database 10, the Y-chromosome genetic variation detection step S111 performs general processes, such as genome/DNA analysis, mapping to a standard genome map, and variant calling. In this case, the embodiments are not limited for a specific configuration for algorithms and devices for analyzing the genome, chromosome, and DNA or algorithms and devices for extracting genetic variation information later.

The Y-chromosome marker designation step S112 may mutually compare the genetic variations detected through the Y-chromosome genetic variation detection step S111 while Y-chromosomes and the surname information for the corresponding man are steadily accumulated in the Y-chromosome group database 10 and may designate a marker for determining the Y-chromosome depending on the distance on the Y-chromosome map.

For example, a database, i.e., the Y-chromosome group database 10, may be built for the Y-chromosomes for 2,000 men having various surnames and their surnames, and a specific marker may be designated for a corresponding chromosome based on the genetic variation information about the Y-chromosome. In this case, the marker has all frequencies between 0.01% and 99% of the occurrence frequency in the population, and the markers for the cases where all people have and it occurs in very few cases, e.g., 0.01%, may be excluded.

The Y-chromosome clustering step S113 may group the Y-chromosomes for the ancient and modern males and, if a specific surname for the males grouped according to the genetic variations in Y-chromosome occupy ⅓ or more of all the surnames, specify the corresponding group as a Y-chromosome group and store it in the Y-chromosome group database 10.

In the instant embodiment, the Y-chromosome group means a group clustered when a general clustering algorithm and phylogenetic diagram are drawn in the population, and when there is a group in which the surname of the males belonging to the cluster occupies 33% or more among such groups, the group may be specified as the Y-chromosome group. Here, 33% means that a specific surname occupies ⅓ of the population. Besides, depending on each surname, an empirically adjusted number, which is variable, such as 40% or 50%, may be used. As such, without limited to a specific reference value, it is important to be able to cluster into empirically most of Y-chromosome groups.

Meanwhile, if Y-chromosome groups are unable to be clearly distinguished per surname (i.e., when an overlap occurs between the surnames), such a process may be performed as to designate a representative indicator (one or a few) for each surname, positioned in the center or middle of each per-surname distribution to clearly process the position of each surname on the cluster.

The male genetic surname determination step s120 may map the genetic marker for determining the male client's Y-chromosome to the genetic marker of the Y-chromosome in each Y-chromosome group stored in the Y-chromosome group database 10 to thereby calculate the genetic distance and determine (actually predict or estimate) that the surname of the Y-chromosome group having the genetic distance closest to the male client's Y-chromosome is the male client's genetic surname. To that end, the male genetic surname determination step S120 may include a genetic surname similarity calculation step S121 and a genetic surname prediction step S122 as shown in FIG. 7.

The genetic surname similarity calculation step S121 may map the genetic marker for determining the male client's Y-chromosome to the genetic marker of the Y-chromosome in each Y-chromosome group stored in the Y-chromosome group database 10 to thereby calculate each genetic distance, score the similarity between the male client's and the surnames belonging to each Y-chromosome group, and provide a numerical result. Here, the genetic surname similarity calculation step S121 may use an algorithm (including all common algorithms for calculating genetic distance) that maps the marker for determining the male client's Y-chromosome based on the Y-chromosome group database 10 and calculates the distance closest to the Y-chromosome group in the population (e.g., 2,000 people). According to the general concept of the algorithm for calculating genetic distance, each male client has a genetic distance of Y-chromosome for each surname. The genetic distance of Y-chromosome may thus be represented as a score indicating how close to the Y-chromosome group each individual's surname is.

In this case, according to the general concept of calculating the genetic distance of Y-chromosome group, each Y-chromosome group has a database for migration of Y-chromosome group through comparison between a Y-chromosome group database for ancient males and a Y-chromosome group database for international males. Such migration database may become a distinguishing marker indicating how males in the Y-chromosome group have migrated from Africa.

The genetic surname prediction step S122 may predict the surname of the Y-chromosome group having the highest similarity score to the Y-chromosome of the male client through the genetic surname similarity calculation step S121, as the male client's genetic surname.

The male genetic surname information providing step S130 may provide the male client with genetic surname information including at least one of social information, historical information, emotional information, and physical information about the modern or past males having the genetic surname determined through the male genetic surname determination step S120.

More specifically, the male genetic surname information providing step S130 may provide each of social information about figures known to the public among the males belonging to the genetic surname determined through the male genetic surname determination step S120, historical information including past migration and settlement route information about the males belonging to the surname discovered in the Y-chromosome group of the corresponding genetic surname, emotional information including the personality of the males belonging to the genetic surname, and physical information including body characteristics, blood type and disease about the males belonging to the genetic surname.

The male genetic surname information providing step S130 may provide various pieces of statistical information, such as famous figures (historically) in the present or past among the males belonging to the genetic surname determined through the male genetic surname determination step S120, historical and social information about the figures, and distribution of surnames discovered in the Y-chromosome group. Further, the male genetic surname information providing unit 130 may provide entertainment information related to anecdotes or characteristics for the surnames of the Y-chromosome group based on the Y-chromosome group. Further, the male genetic surname information providing unit 130 may provide various pieces of information, such as personality, body feature, or blood type of individuals based on the Y-chromosome group. Further, the male genetic surname information providing unit 130 may provide information about the migration or settlement route of ancestors of the Y-chromosome group based on the Y-chromosome group. Such information may be manufactured and provided as image information by way of media through an application and web.

The X-chromosome grouping step S140 may identify the genetic variation of the autosomal, X-chromosomal and mitochondrial DNAs of the males belonging to the Y-chromosome group, combine the autosomal, X-chromosomal and mitochondrial DNAs with the same or most similar distribution patterns of identified genetic variations, specify the combined autosomal, X-chromosomal and mitochondrial DNAs as an X-chromosome group and store it in the X-chromosome group database 20. The X-chromosome grouping step S140 is configured to find females' surnames based on the genetic information about the autosomal, X-chromosomal and mitochondrial DNAs of people belonging to the Y-chromosome group in a state in which the Y-chromosome group database 10 is built. The X-chromosome grouping step S140 has substantially the same configuration as that of the Y-chromosome grouping step S110, and a detailed description thereof is thus omitted.

The female genetic surname determination step S150 may map the genetic marker for determining the female client's X-chromosome to the genetic marker of the X-chromosome in each X-chromosome group stored in the X-chromosome group database 20 to thereby calculate the genetic distance and determine that the surname of the X-chromosome group having the genetic distance closest to the client's X-chromosome is the client's genetic surname. The female genetic surname determination step S150 has substantially the same configuration as that of the male genetic surname determination step S120, and a detailed description thereof is thus omitted.

Accordingly, the male genetic surname information providing step S130 may provide the client with genetic surname information including at least one of social information, historical information, emotional information and physical information about the modern or ancient males determined through the female genetic surname determination step S150 and the configuration therefor is performed in the same manner as the operation of the male genetic surname information providing step S130 described above.

An embodiment of the present invention may define the Y-chromosome group where a male himself belongs and sub groups thereof, precisely calculate the degree of genetic variation to the Y-chromosome to thereby infer the genetic surname closest to the male, such as siblings, ancestors, cousins, first cousins once removed, or first cousins four times removed, and provide a female's surname by comparing the autosomal, X-chromosomal, and mitochondrial DNA information possessed by the Y-chromosome-based genetic surname group and the female's autosomal, X-chromosomal, and mitochondrial DNA information, using variation information about the autosomal, X-chromosomal, and mitochondrial DANs present in both the male and female.

What is claimed is:

1. A system for providing genetic surname information using genomic information, comprising:
a Y-chromosome grouping unit identifying genetic variations of Y-chromosomes for ancient and modern males in each area or country, grouping the Y-chromosomes for the ancient and modern males based on the identified genetic variations, and if a specific surname for the males grouped according to the genetic variations is shown in a predetermined proportion or more, specifying a corresponding group as a Y-chromosome group and storing the Y-chromosome group in a Y-chromosome group database;
a male genetic surname determination unit mapping a genetic marker for determining a male client's Y-chromosome to a genetic marker in a Y-chromosome in each Y-chromosome group stored in the Y-chromosome group database to thereby calculate a genetic distance and determining that a surname of a Y-chromosome group having the genetic distance closest to the male client's Y-chromosome is the male client's genetic surname; and
a male genetic surname information providing unit providing the male client with genetic surname information including at least one of social information, historical information, emotional information, and physical information about the modern or ancient males having the genetic surname determined through the male genetic surname determination unit.

2. The system of claim 1, wherein the Y-chromosome grouping unit includes:
a Y-chromosome genetic variation detection unit analyzing the genetic information about the Y-chromosomes for the ancient and modern males in each area or country, comparing sequences of the Y-chromosomes for which the genetic information has been analyzed, and detecting genetic variations of the Y-chromosomes from a result of the comparison;
a Y-chromosome marker designation unit mutually comparing the genetic variations detected through the Y-chromosome genetic variation detection unit and designating a marker for determining a Y-chromosome according to a distance of the Y-chromosome on a map; and
a Y-chromosome clustering unit grouping the Y-chromosomes for the ancient and modern males and, if the specific surname for the males grouped according to the genetic variations of the Y-chromosomes occupies ⅓ or more of all surnames, specifying the corresponding group as the Y-chromosome group, and storing the Y-chromosome group in the Y-chromosome group database.

3. The system of claim 1, wherein the male genetic surname determination unit includes:
a genetic surname similarity calculation unit mapping the genetic marker for determining the male client's Y-chromosome to the genetic marker of the Y-chromosome in each Y-chromosome group stored in the Y-chromosome group database to thereby calculate the genetic distance, and scoring a similarity between the male client and the surnames belonging to each Y-chromosome group; and
a genetic surname prediction unit predicting a surname of a Y-chromosome group having a highest similarity score with the male client's Y-chromosome through the genetic surname similarity calculation unit, as the male client's genetic surname.

4. The system of claim 1, wherein the male genetic surname information providing unit provides social information about a figure known to the public among the males belonging to the genetic surname determined through the male genetic surname determination unit, historical information including past migration and settlement route information about the males belonging to the surname discovered in the Y-chromosome group of the genetic surname, emotional information including personality of the males belonging to the genetic surname, and physical information including body features, blood types, and diseases of the males belonging to the genetic surname.

5. The system of claim 1, further comprising:
an X-chromosome grouping unit identifying genetic variations of autosomal, X-chromosomal and mitochondrial DNAs of the males belonging to the Y-chromosome group, combining the autosomal, X-chromosomal and mitochondrial DNAs with the same or most similar distribution patterns of the identified genetic variations, specifying the combined autosomal, X-chromosomal and mitochondrial DNAs as an X-chromosome group, and storing the X-chromosome group in an X-chromosome group database; and
a female genetic surname determination unit mapping a genetic marker for determining a female client's X-chromosome to a genetic marker in an X-chromosome in each X-chromosome group stored in the X-chromosome group database to thereby calculate a genetic distance and determining that a surname of an X-chromosome group having the genetic distance closest to the client's X-chromosome is the client's genetic surname, wherein the male genetic surname information providing unit provides the client with genetic surname information including at least one of social information, historical information, emotional information, and physical information about the modern or ancient males having the genetic surname determined through the female genetic surname determination unit.

6. A method for providing genetic surname information using genomic information, comprising:
a Y-chromosome grouping step identifying genetic variations of Y-chromosomes for ancient and modern males in each area or country, grouping the Y-chromosomes for the ancient and modern males based on the identified genetic variations, and if a specific surname for the males grouped according to the genetic variations is shown in a predetermined proportion or more, specifying a corresponding group as a Y-chromosome group and storing the Y-chromosome group in a Y-chromosome group database, by a Y-chromosome grouping unit;
a male genetic surname determination step mapping a genetic marker for determining a male client's Y-chromosome to a genetic marker in a Y-chromosome in each Y-chromosome group stored in the Y-chromosome group database to thereby calculate a genetic distance and determining that a surname of a Y-chromosome group having the genetic distance closest to the male client's Y-chromosome is the male client's genetic surname, by a male genetic surname determination unit; and
a male genetic surname information providing step providing the male client with genetic surname information including at least one of social information, historical information, emotional information, and physical information about the modern or ancient males having the genetic surname determined through the male genetic surname determination step, by a male genetic surname information providing unit.

7. The method of claim 6, wherein the Y-chromosome grouping unit step includes:
a Y-chromosome genetic variation detection step analyzing the genetic information about the Y-chromosomes for the ancient and modern males in each area or country, comparing sequences of the Y-chromosomes for which the genetic information has been analyzed, and detecting genetic variations of the Y-chromosomes from a result of the comparison;
a Y-chromosome marker designation step mutually comparing the genetic variations detected through the Y-chromosome genetic variation detection step and designating a marker for determining a Y-chromosome according to a distance of the Y-chromosome on a map; and
a Y-chromosome clustering step grouping the Y-chromosomes for the ancient and modern males and, if the specific surname for the males grouped according to the genetic variations of the Y-chromosomes occupies ⅓ or more of all surnames, specifying the corresponding group as the Y-chromosome group, and storing the Y-chromosome group in the Y-chromosome group database.

8. The method of claim 6, wherein the male genetic surname determination step includes:
a genetic surname similarity calculation step mapping the genetic marker for determining the male client's Y-chromosome to the genetic marker of the Y-chromosome in each Y-chromosome group stored in the Y-chromosome group database to thereby calculate the genetic distance, and scoring a similarity between the male client and the surnames belonging to each Y-chromosome group; and
a genetic surname prediction step predicting a surname of a Y-chromosome group having a highest similarity score with the male client's Y-chromosome through the genetic surname similarity calculation step, as the male client's genetic surname.

9. The method of claim 6, wherein the male genetic surname information providing step provides social information about a figure known to the public among the males belonging to the genetic surname determined through the male genetic surname determination step, historical information including past migration and settlement route information about the males belonging to the surname discovered in the Y-chromosome group of the genetic surname, emotional information including personality of the males belonging to the genetic surname, and physical information including body features, blood types, and diseases of the males belonging to the genetic surname.

10. The method of claim 6, further comprising:
an X-chromosome grouping step identifying genetic variations of autosomal, X-chromosomal and mitochondrial DNAs of the males belonging to the Y-chromosome group, combining the autosomal, X-chromosomal and mitochondrial DNAs with the same or most similar distribution patterns of the identified genetic variations, specifying the combined autosomal, X-chromosomal and mitochondrial DNAs as an X-chromosome group, and storing the X-chromosome group in an X-chromosome group database, by an X-chromosome grouping unit; and
a female genetic surname determination step mapping a genetic marker for determining a female client's X-chromosome to a genetic marker in an X-chromosome in each X-chromosome group stored in the X-chromosome group database to thereby calculate a genetic distance and determining that a surname of an X-chromosome group having the genetic distance closest to the client's X-chromosome is the client's genetic surname, by a female genetic surname determination unit, wherein the male genetic surname information providing step provides the client with genetic surname information including at least one of social information, historical information, emotional information, and physical information about the modern or ancient males having the genetic surname determined through the female genetic surname determination step.
